(19) United States
(12) Patent Application Publication
(10) Pub. No.: US 2026/0243749 A1
(43) Pub. Date: Aug. 20, 2026

(54) REACTION TUBE FOR WATER QUALITY MEASUREMENT DEVICE INCLUDING CRF TUBE

(71) Applicants: ATT CO., LTD., Cheonan-si, Chungcheongnam-do (KR); ANALTEC TMS CO., LTD., Daegu (KR)

(72) Inventor: Jae Cheol KIM, Daegu (KR)

(73) Assignees: ATT CO., LTD., Cheonan-si, Chungcheongnam-do (KR); ANALTEC TMS CO., LTD., Daegu (KR)

(21) Appl. No.: 19/160,671

(22) PCT Filed: Mar. 8, 2024
(86) PCT No.: PCT/KR2024/003035

§ 371 Date: Aug. 28, 2025

(30) Foreign Application Priority Data
Mar. 8, 2023 (KR) 10-2023-0030264

(51) Int. Cl. G01N 33/18; C02F 1/00; C02F 101/12

(52) U.S. Cl. CPC G01N 33/18; C02F 1/008; C02F 2101/12

(57) ABSTRACT

A reaction tube for a water quality measurement device including a chlorine removal filter (CRF) pipe, includes: a CRF tube portion in which a sample is introduced and stored and gas is discharged to a position higher than a water level of the sample; an induction portion that surrounds the CRF tube portion and is provided such that the discharged gas is discharged to one side again; and an induction passage that is provided between the CRF tube portion and the induction portion so that the gas is movable along the induction portion.

### CROSS-REFERENCE TO PRIOR APPLICATIONS

This Application is a National Stage Patent Application of PCT International Application No. PCT/KR 2024/003035 (filed on March 8, 2024), which claims priority to Korean Patent Application No. 10-2023-0030264 (filed on March 8, 2023), which are all hereby incorporated by reference in their entirety.

### BACKGROUND

The technology disclosed in the present disclosure relates to a reaction tube, and more specifically, to a reaction tube for water quality measurement device including a chlorine removal filter (CRF) tube in which water is vaporized and reacts with a catalyst in a water quality measurement device for measuring water quality.

Korean Patent No. 10-2231380 (published on Mar. 24, 2021, “Total organic carbon measuring device capable of continuous sample injection”) discloses a total organic carbon measuring device capable of continuous sample injection. The total organic carbon measuring device capable of continuous sample injection includes a sample storage unit in which a measurement target sample for measuring the degree of water pollution is stored, a combustion unit for heating the sample stored in the sample storage unit, a multi-valve for pumping to transfer the sample stored in the sample storage unit to the combustion unit, a sample injection unit for injecting a set amount of the sample transferred from the multi-valve into the combustion unit, and a measuring unit for measuring the total organic carbon content in gas vaporized through the combustion unit to determine the degree of water pollution. In the total organic carbon measuring device, the sample injection unit is formed of a sliding valve, and is configured to inject a preset amount of sample supplied from the multi-valve into the combustion unit at a first preset position in a state of being in communication with the combustion unit, and to discharge a preset amount of sample supplied from the multi-valve to the outside while sliding from the first preset position to a second preset position, and the sample is controlled to be continuously injected into or discharged from the combustion unit within a preset cycle.

In addition, Korean Patent No. 10-2187689 (published on Dec. 7, 2020, “TOC Measurement Crucible Furnace Structure”) includes a TOC measurement crucible furnace structure that heats introduced measurement water, including a heating housing having an operating space formed inside, a heating pipe that is installed penetrating the heating housing and receives and heats the measurement water, and a heater unit that heats the heating pipe, the heating housing is further provided with an elevating heating temperature control means to detect temperature of the heating wire units of the heater unit that heats the heating pipe and then adjust the temperature, and the elevating heating temperature control means includes an elevating cylinder whose lower end is connected and fixed to the inner bottom surface of the heating housing, a bracket unit including a detection bracket and a blower bracket that are fixed to an elevating shaft of the elevating cylinder and are raised and lowered by the driving of the elevating cylinder, a temperature detection sensor installed on the detection bracket, raised and lowered by the driving of the elevating cylinder, and detecting the temperature of the heating wire units, and a blower fan installed on the blower bracket, raised or lowered by the driving of the elevating cylinder, and supplying wind to the heating wire units.

The reaction tube used in the conventional water quality measurement device introduced above is a total organic carbon measuring device that applies a method of converting into carbon dioxide using an oxidation reactor as described in Korean Patent No. 10-1097874 (published on Dec. 23, 2011, “Non-catalytic thermal combustion total organic carbon content measuring device using ceramic ball”), which includes a reactor 20, a cylindrical protection tube 22 inserted into the interior of the reactor 20 and having a sieve 24 installed at a lower end thereof, ceramic balls 26 filling the cylindrical protection tube 22, a heater for controlling the temperature to 1,200 ±200 degrees Celsius, and a temperature control unit for supplying current to the heater.

Korean Patent No. 10-2013-0036037 (published on Apr. 9, 2013, “Non-catalytic thermal combustion oxidation device for water quality analysis device”) discloses a technology including a reaction tube 2 including a cylindrical reactor 20 having an open upper portion, extending downwardly, and having an empty space 200 inside, narrowing to a predetermined diameter near the lower end portion, and having a tube having the predetermined diameter and extending downward by a predetermined length, a protection tube 22 which is a cylindrical pipe whose outer wall is in close contact with the inner wall of the cylindrical reactor, and an inner tube 24 that is configured to be L/5 or more and less than L/2 when a length of the protection tube 22 is L, is installed by being inserted into the inside of the protection tube 22 at a certain interval from the inner wall, has a plurality of through holes 240 on the surface, has a plurality of through holes 241 at the upper end and is sealed, and has a central portion 242 at the lower end that has an outer diameter corresponding to the inner diameter of the protection tube 22 and is open, and has a filter mesh 244 with a plurality of holes 243 perforated from the central portion 242 to the outer edge.

### SUMMARY

Conventional reaction tubes for water quality measurement devices including chlorine removal filter (CRF) tube have a problem in that foreign substances or salts contained in the sample remain and impede the movement of vaporized gas, thereby shortening the lifespan of the reaction tube for water quality measurement devices including the chlorine removal filter (CRF) tube.

Accordingly, an object of the technology disclosed in the present disclosure is to provide a reaction tube for water quality measurement devices including a chlorine removal filter (CRF) tube that can extend the lifespan of the reaction tube for water quality measurement devices including a chlorine removal filter (CRF) tube by preventing the movement of gas from being stagnant even when foreign substances or salts contained in the sample remain.

One embodiment discloses a reaction tube for a water quality measurement device including a chlorine removal filter (CRF) pipe.

The reaction tube may include: a CRF tube portion 100 in which a sample is introduced and stored and gas is discharged to a position higher than a water level of the sample; an induction portion 200 that surrounds the CRF tube portion 100 and is provided such that the discharged gas is discharged to one side again; and an induction passage 300 that is provided between the CRF tube portion 100 and the induction portion so that the gas is movable along the induction portion 200.

The CRF tube portion 100 may include a vaporizing body 110 provided in the form of a sealed tube, a sample passage 120 provided so that the sample is introduced to one side surface of the vaporizing body 110, and a gas passage 130 provided on the other side surface of the vaporizing body 110 and provided at a position higher than the water level of the introduced sample.

The induction portion 200 may be provided in the form of a tube with one end closed and provided in a size such that the vaporizing body 110 can be positioned inside, and the induction passage 300 may be provided spaced apart from the vaporizing body 110.

The induction portion may be provided in the form of a tube and provided longer than the vaporizing body 110, and an inner surface of the induction portion may be provided to be in contact with a surface of the vaporizing body 110, and the induction passage 300 may be provided longitudinally on the inner surface of the induction portion 200 facing the gas passage 120.

The CRF tube portion may include a vaporizing body 110 provided in the form of a tube with one end closed, a heat sink 140 that seals an open portion of the vaporizing body 110, a sample passage 120 provided so that the sample is introduced into the heat sink 140, and a gas passage 130 provided on the other side surface of the vaporizing body 110 and provided at a position higher than a water level of the introduced sample.

The induction portion 200 may be provided in a form of a tube and provided in a size such that the vaporizing body 110 is located inside the induction portion 200, the heat sink 140 may be provided to further seal one open side of the induction portion 200, and include a first coupling groove 141 provided for inserting the vaporizing body 110, and a second coupling groove 142 provided in a form that surrounds the first coupling groove 141, the induction portion 200 being inserted into the second coupling groove 142 so that a surface of the vaporizing body 110 and an inner surface of the induction portion 200 are separated from each other to provide the induction passage 300.

The reaction tube may further include an inlet passage 400 connected to the induction passage 300 and through which external air is introduced.

The CRF tube portion 100 may include a vaporizing body 110 provided in the form of a tube with one end closed, a heat sink 140 that seals an open portion of the vaporizing body 110, a sample passage 120 provided so that the sample is introduced into the heat sink 140, and a gas passage 130 provided on the other side surface of the vaporizing body 110 and provided at a position higher than a water level of the introduced sample, the induction portion 200 may be provided in a form of a tube and provided in a size such that the vaporizing body is located inside the induction portion 200, the heat sink 140 may be provided to further seal one open side of the induction portion 200, and include a first coupling groove 141 provided for inserting the vaporizing body 110, and a second coupling groove 142 provided in a form that surrounds the first coupling groove 141, the induction portion 200 being inserted into the second coupling groove 142 so that a surface of the vaporizing body 110 and an inner surface of the induction portion are separated from each other to provide the induction passage 300, the first coupling groove 141 may be formed deeper than the second coupling groove 142, and the inlet passage 400 may penetrate a side surface of the heat sink 140 and be connected to the induction passage 300 toward the second coupling groove 142.

According to the reaction tube for a water quality measurement device including a chlorine removal filter (CRF) tube disclosed in the present disclosure, it is possible to maintain smooth movement of gas for a long time by preventing salt particles including foreign substances included in a sample or elements generated when the sample is vaporized from accumulating at the lower portion of the vaporizing body 110, thereby preventing salt particles including foreign substances or chlorine generated when the sample is vaporized from accumulating in the gas passage 120 or the induction passage 300 or the induction portion 200 through which gas moves. According to the reaction tube for a water quality measurement device including a chlorine removal filter (CRF) tube including an inlet passage 400 according to another embodiment described above, it is possible to shorten the water quality measurement time by promoting smooth movement of gas of a vaporized sample discharged to the gas passage 130 by generating a flow in which air supplied from one side of the induction portion 200 moves along the surface of the vaporizing body 110 to the open portion of the induction portion 200.

The foregoing provides only selective concepts in a simplified form for matters described in more detail later. This content is not intended to limit the main features or essential features of the claims or to limit the scope of the claims.

### BRIEF DESCRIPTION OF THE DRAWINGS

FIG. 1 is a diagram illustrating a reaction tube for a water quality measurement device including a CRF tube disclosed in the present disclosure.

FIG. 2 is a diagram illustrating a longitudinal section of the reaction tube for the water quality measurement device including the CRF tube disclosed in FIG. 1.

FIG. 3 is a diagram illustrating a functional relationship of the reaction tube for the water quality measurement device including the CRF tube disclosed in FIG. 2.

FIG. 4 is a diagram illustrating another example of an induction portion disclosed in the present disclosure.

FIG. 5 is a diagram illustrating a longitudinal section according to another embodiment of a reaction tube for the water quality measurement device including the CRF tube disclosed in the present disclosure.

FIG. 6 is a diagram illustrating another example of a heat sink disclosed in the present disclosure.

FIG. 7 is a cross-sectional diagram of another embodiment of the reaction tube for the water quality measurement device including the CRF tube disclosed in the present specification.

### DETAILED DESCRIPTION

Hereinafter, embodiments disclosed in the present specification will be described in detail with reference to the drawings. Unless otherwise specified in the text, similar reference numerals in the drawings represent similar components. The exemplary embodiments described in the detailed description, drawings, and claims are not intended to be limiting, and other embodiments may be utilized and other changes may be made without departing from the spirit or scope of the technology disclosed herein. It will be readily understood by those skilled in the art that the components of the present disclosure, that is, the components generally described herein and illustrated in the drawings, may be arranged, configured, combined, and designed in various different configurations, all of which are expressly contemplated and form a part of the present disclosure. In the drawings, the width, length, thickness, or shape of the components may be exaggerated in order to clearly express various layers (or films), regions, and shapes.

When one component is referred to as “included” to another component, it may include cases where the one component is directly included to the other component, as well as cases where additional components are interposed between them.

When one component is referred to as “provided” to another component, it may include cases where the one component is directly provided to the other component, as well as cases where additional components are interposed between them.

Since the description of the disclosed technology is merely an embodiment for structural or functional explanation, the scope of the disclosed technology should not be construed as being limited by the embodiments described in the text. In other words, since the embodiments can be variously modified and can have various forms, the scope of the disclosed technology should be understood to include equivalents that can realize the technical idea.

The singular expression should be understood to include the plural expression unless the context clearly indicates otherwise, and the terms “include” or “have” are intended to specify the presence of a feature, number, step, operation, component, part, or combination thereof implemented, but should be understood not to preclude the possibility of the presence or addition of one or more other features, numbers, steps, operations, components, parts, or combinations thereof.

All terms used herein, unless otherwise defined, have the same meaning as generally understood by a person skilled in the art to which the disclosed technology belongs. Terms defined in commonly used dictionaries should be interpreted as having a meaning consistent with the contextual meaning of the relevant technology, and should not be interpreted as having an ideal or excessively formal meaning unless explicitly defined in this application.

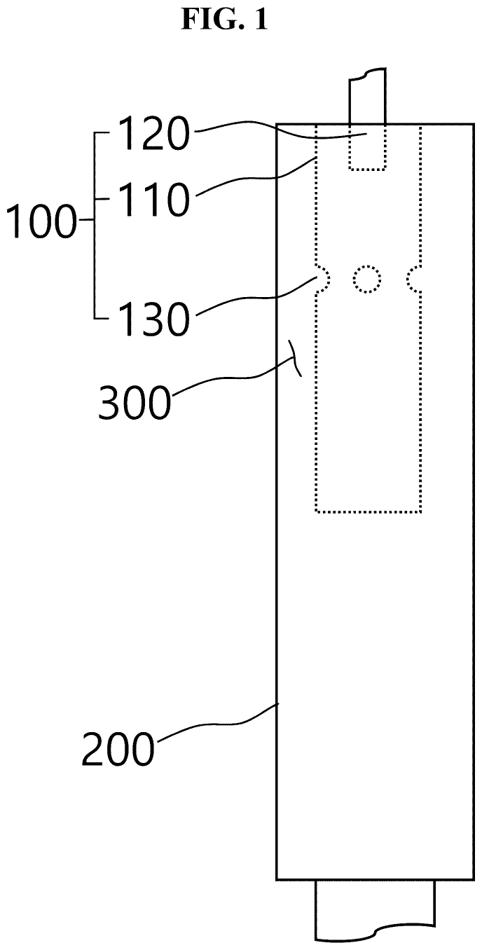

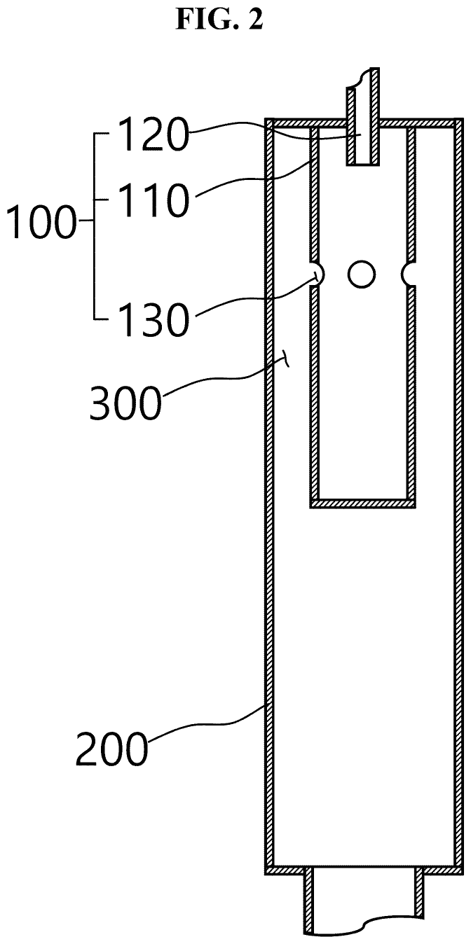

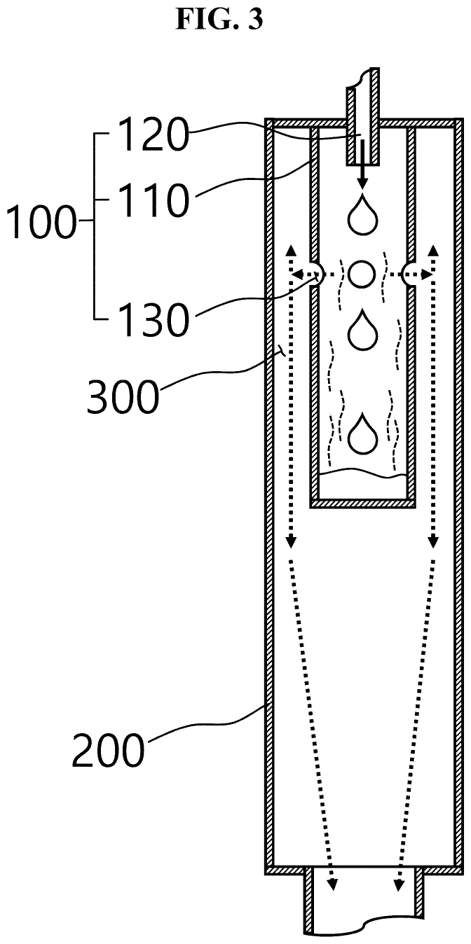

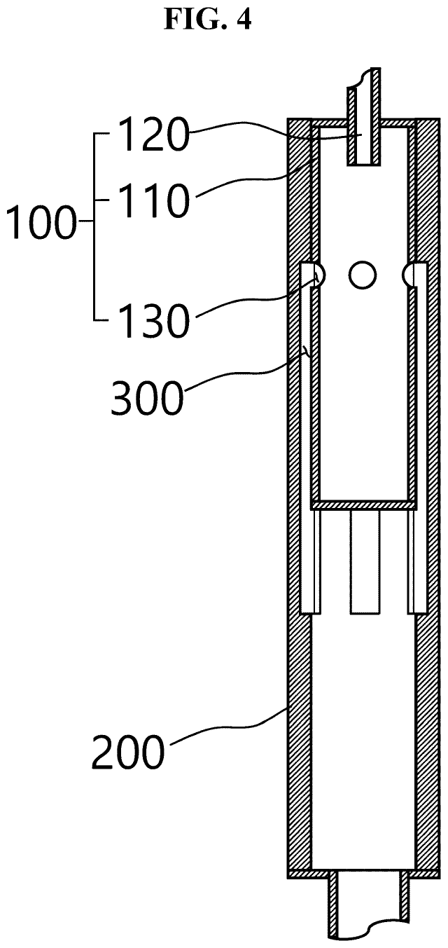

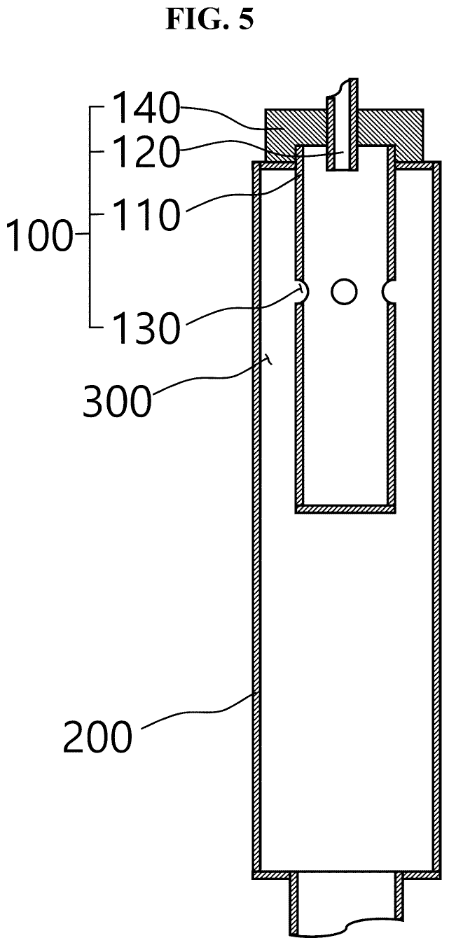

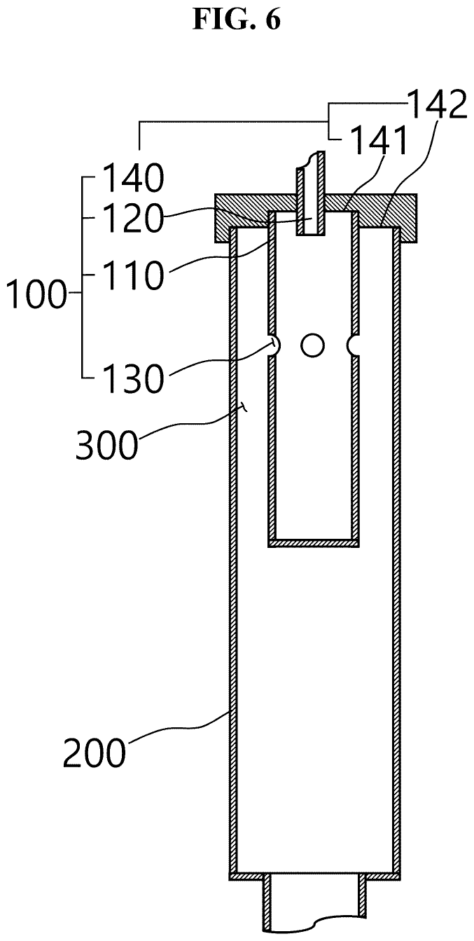

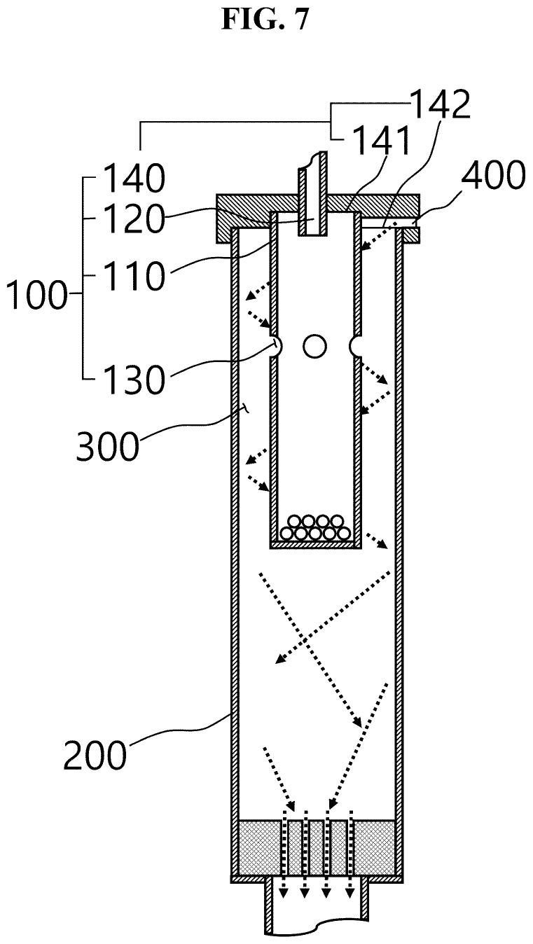

FIG. 1 of the drawings attached to the present disclosure is a diagram illustrating a reaction tube for a water quality measurement device including a CRF tube disclosed in the present disclosure. FIG. 2 is a diagram illustrating a longitudinal section of the reaction tube for the water quality measurement device including the CRF tube disclosed in FIG. 1. FIG. 3 is a diagram illustrating a functional relationship of the reaction tube for the water quality measurement device including the CRF tube disclosed in FIG. 2. FIG. 4 is a diagram illustrating another example of an induction portion disclosed in the present disclosure. FIG. 5 is a diagram illustrating a longitudinal section according to another embodiment of a reaction tube for the water quality measurement device including the CRF tube disclosed in the present disclosure. FIG. 6 is a diagram illustrating another example of a heat sink disclosed in the present disclosure. FIG. 7 is a cross-sectional diagram of another embodiment of the reaction tube for the water quality measurement device including the CRF tube disclosed in the present specification.

Hereinafter, the present disclosure will be described in more detail with reference to the drawings attached to the present specification.

The present disclosure relates to a reaction tube for a water quality measurement device including a chlorine removal filter (CRF) tube, which includes a CRF tube portion 100, an induction portion 200, and an induction passage 300. In the reaction tube for the water quality measurement device including the chlorine removal filter (CRF) tube, a sample is heated and vaporized in the CRF tube portion 100. The vaporized gas is discharged from the CRF tube portion 100 and guided to the induction portion 200 by the induction passage 300 and moves along the induction portion 200. The heating of the CRF tube portion 100 may be performed directly or indirectly by a conventional heating wire, as disclosed in conventional technologies.

The CRF tube portion 100 is provided in a form in which a sample can be introduced and stored. The sample is water for measuring water quality. The CRF tube portion 100 may be provided so that the gas of the water heated to a position higher than the water level of the sample is discharged.

For example, referring to FIGS. 1 to 4, a vaporizing body 110 may be provided in a sealed tube shape. The vaporizing body 110 may be positioned vertically. A sample passage 120 may be provided on one side of the vaporizing body 110 so that the sample may be introduced. The sample passage 120 may be provided by a pipe penetrating the upper surface of the vaporizing body 110 through which the sample is moved. The gas passage 130 may be provided on the other side of the vaporizing body 110. The gas passages 130 may be provided on the front/rear/left/right sides of the vaporizing body 110, respectively. The gas passages 130 may be provided by forming a hole at a position higher than the water level of the introduced sample. The gas passage 130 may also be provided in the middle part of the longitudinal direction of the vaporizing body 110.

As another example referring to FIG. 5, the CRF tube portion 100 may include the vaporizing body 110, the sample passage 120, the gas passage 130, and a heat sink 140. The vaporizing body 110 may be provided in a tube shape with one end blocked. When the vaporizing body 110 is positioned vertically, the lower portion may be blocked and the upper portion may be open. The heat sink 140 may be provided with a typical heat-dissipating material. The heat sink 140 suppresses the movement of heat of the heated vaporizing body 110. The heat sink 140 may be provided in a shape that seals the open portion of the vaporizing body 110. The sample passage 120 may be provided so that the sample may be introduced into the heat sink 140. The sample passage 120 may be provided by a pipe through which the sample is moved penetrating the heat sink 140. The gas passage 130 may be provided on the other side of the vaporizing body 110. The gas passage 130 may be provided by forming a hole at a position higher than the water level of the introduced sample. The gas passage 130 may also be provided at the middle portion of the length direction of the vaporizing body 110.

Meanwhile, referring to FIGS. 6 and 7, the heat sink 140 may include a first coupling groove 141 and a second coupling groove 142 to close the open portions of the vaporizing body 110 and the induction portion 200, respectively. The heat sink 140 including the first coupling groove 141 and the second coupling groove 142 may be implemented in a state where the vaporizing body 110 is provided in a tube shape with one end closed, and the induction portion 200 is provided in a tube shape and is provided larger than the vaporizing body 110. The heat sink 140 may be provided in a form that closes the open portion of the vaporizing body 110 and be provided to further close the open one end of the induction portion 200. To explain in more detail, the heat sink 140 may be provided in a size and shape that can cover one open side of the vaporizing body 110, and may include the first coupling groove 141 into which the open side of the vaporizing body 110 is inserted. The heat sink 140 may be provided so as to protrude further laterally so as to cover one open side of the induction portion 200 in a shape that surrounds the first coupling groove 141. The heat sink 140 may be provided with the second coupling groove 142 so that one open side of the induction portion 200 is inserted in a shape that surrounds the first coupling groove 141. The heat sink 140 is provided so that the induction portion 200 is inserted into the second coupling groove 142 so that the surface of the vaporizing body 110 and the inner surface of the induction portion 200 are separated to form the induction passage 300. The induction passage 300 allows gas to move between the CRF tube portion 100 and the induction portion 200.

The induction portion 200 referring to the attached drawing may be provided to surround the CRF tube portion 100. That is, the induction portion 200 is provided in a size such that the vaporizing body 110 can be positioned inside. The induction portion 200 is provided so that the gas discharged from the gas passage 130 is discharged again to one side.

The induction portion 200 according to an example referring to FIGS. 1 to 3 and 5 to 7 may be provided in the form of a tube with one end blocked. The induction portion 200 may be positioned so that an open portion is positioned in the direction in which the bottom surface of the vaporizing body 110 faces. The induction portion 200 induces the movement of gas so that the gas discharged from the gas passage 130 moves to the open portion. The induction portion 200 may be provided to be larger than the vaporizing body 110. The inner surface of the induction portion 200 may be positioned so that the induction passage 300 is provided so that the inner surface is spaced apart from the vaporizing body 110. A pipe through which a sample is moved is passed through the upper surface of the induction portion 200. The induction passage 300 allows gas to move between the CRF tube portion 100 and the induction portion 200.

As the example referring to FIG. 4, the induction portion 200 may be provided in a tube shape. The induction portion 200 may be provided longer than the vaporizing body 110. The induction portion 200 may be provided so that the inner surface thereof is in contact with the surface of the vaporizing body 110. In this case, the induction passage 300 may be provided by forming a longitudinal groove on the inner surface of the induction portion 200 facing the gas passage 120. The length of the induction passage 300 may be provided longer than the length of the vaporizing body 110. The induction passage 300 is connected to the gas passage 120. One side of the induction portion 200 is blocked by the vaporizing body 110, and the other side is open. The reaction tube for the water quality measurement device including the chlorine removal filter (CRF) tube according to the embodiment described above is installed inside the water quality measurement device. The reaction tube for the water quality measurement device including the chlorine removal filter (CRF) tube moves a sample to a sample passage 120 by the water quality measurement device. The moved sample is stored in the lower portion of the vaporizing body 110. The vaporizing body 110 is heated by the water quality measurement device to vaporize the sample. The gas of the vaporized sample is discharged to an induction passage 330 through the gas passage 130. The gas discharged to the induction passage 330 is moved to an open portion on one side of an induction portion 200. The gas moved through the induction portion 200 is reacted by a catalyst (not illustrated).

According to the reaction tube for the water quality measurement device including the chlorine removal filter (CRF) tube disclosed in the present disclosure described above, it is possible to maintain smooth movement of gas for a long time by preventing salt particles including foreign substances included in the sample or elements generated when the sample is vaporized from accumulating at the lower portion of the vaporizing body 110, thereby preventing salt particles including foreign substances or chlorine generated when the sample is vaporized from accumulating in the gas passage 120 or the induction passage 300 or the induction portion 200 through which gas moves.

Meanwhile, as illustrated in FIG. 7, a heat preservation portion made of ceramic material may be further included inside the CRF tube portion 100. The heat preservation portion may be provided in a bead shape. The heat preservation portion is provided below the height of the gas passage 120. The heated heat preservation portion can promote vaporization of the sample.

Meanwhile, as in FIG. 7, a conventional catalyst that reacts with gas may be further included inside the induction portion 200.

The reaction tube for the water quality measurement device including the chlorine removal filter (CRF) tube according to another embodiment may further include an inlet passage 400. The inlet passage 400 promotes a flow in which air is introduced and gas is smoothly moved to the open portion of the induction portion 200 through the induction passage 300. That is, the inlet passage 400 is connected to the induction passage 300 to allow external air to be introduced. For example, the inlet passage 400 may be provided by forming a hole (not illustrated) on one side of the induction portion 200. The induction portion 200 may be provided to be connected to the induction passage 300.

As another example referring to FIG. 7, the inlet passage 400 may be provided on the side of the heat sink 140. In this case, the CRF tube portion 100 may include the vaporizing body 110 provided in the form of a tube with one end closed, the heat sink 140 that seals an open portion of the vaporizing body 110, the sample passage 120 provided so that the sample is introduced into the heat sink 140, and the gas passage 130 provided on the other side surface of the vaporizing body 110 and provided at a position higher than a water level of the introduced sample, the induction portion 200 is provided in a form of a tube and provided to be larger than the vaporizing body, the heat sink 140 is provided to further seal one open side of the induction portion 200, and include the first coupling groove 141 provided for inserting the vaporizing body 110, and the second coupling groove 142 provided in a form that surrounds the first coupling groove 141, the induction portion 200 being inserted into the second coupling groove 142 so that the surface of the vaporizing body 110 and an inner surface of the induction portion are separated from each other to provide the induction passage 300. The inlet passage 400 may penetrate a side surface of the heat sink 140 and be connected to the induction passage 300 toward the second coupling groove 142.

Meanwhile, the first coupling groove 141 may be provided deeper than the second coupling groove 142.

According to the reaction tube for a water quality measurement device including a chlorine removal filter (CRF) tube including an inlet passage 400 according to another embodiment described above, it is possible to shorten the water quality measurement time by promoting smooth movement of gas of a vaporized sample discharged to the gas passage 130 by generating a flow in which air supplied from one side of the induction portion 200 moves along the surface of the vaporizing body 110 to the open portion of the induction portion 200.

From the above, it will be understood that various embodiments of the present disclosure have been described for illustrative purposes, and that various modifications are possible without departing from the scope and spirit of the present disclosure. In addition, the various embodiments disclosed are not intended to limit the spirit of the present disclosure, and the true spirit and scope will be set forth in the claims that follow.

1. A reaction tube for a water quality measurement device including a chlorine removal filter (CRF) pipe, the reaction tube comprising:
a CRF tube portion in which a sample is introduced and stored and gas is discharged to a position higher than a water level of the sample;
an induction portion that surrounds the CRF tube portion and is provided such that the discharged gas is discharged to one side again; and
an induction passage that is provided between the CRF tube portion and the induction portion so that the gas is movable along the induction portion.

2. The reaction tube of claim 1, wherein the CRF tube portion includes
a vaporizing body provided in the form of a sealed tube,
a sample passage provided so that the sample is introduced to one side surface of the vaporizing body, and
a gas passage provided on the other side surface of the vaporizing body and provided at a position higher than the water level of the introduced sample.

3. The reaction tube of claim 2, wherein the induction portion is provided in the form of a tube with one end closed and is provided in a size such that the vaporizing body can be positioned inside, and the induction passage is provided spaced apart from the vaporizing body.

4. The reaction tube of claim 2, wherein the induction portion is provided in the form of a tube and is provided longer than the vaporizing body, and an inner surface of the induction portion is provided to be in contact with a surface of the vaporizing body, and the induction passage is provided longitudinally on the inner surface of the induction portion facing the gas passage.

5. The reaction tube of claim 1, wherein the CRF tube portion includes
a vaporizing body provided in the form of a tube with one end closed,
a heat sink that seals an open portion of the vaporizing body,
a sample passage provided so that the sample is introduced into the heat sink, and
a gas passage provided on the other side surface of the vaporizing body and provided at a position higher than a water level of the introduced sample.

6. The reaction tube of claim 5, wherein the induction portion is provided in a form of a tube and provided in a size such that the vaporizing body is located inside the induction portion,
the heat sink is provided to further seal one open side of the induction portion, and includes
a first coupling groove provided for inserting the vaporizing body, and
a second coupling groove provided in a form that surrounds the first coupling groove, the induction portion being inserted into the second coupling groove so that a surface of the vaporizing body and an inner surface of the induction portion are separated from each other to provide the induction passage.

7. The reaction tube of claim 1, further comprising an inlet passage connected to the induction passage and through which external air is introduced.

8. The reaction tube of claim 7, wherein the CRF tube portion includes
a vaporizing body provided in the form of a tube with one end closed,
a heat sink that seals an open portion of the vaporizing body,
a sample passage provided so that the sample is introduced into the heat sink, and
a gas passage provided on the other side surface of the vaporizing body and provided at a position higher than a water level of the introduced sample,
the induction portion is provided in a form of a tube and provided in a size such that the vaporizing body is located inside the induction portion,
the heat sink is provided to further seal one open side of the induction portion, and includes
a first coupling groove provided for inserting the vaporizing body, and
a second coupling groove provided in a form that surrounds the first coupling groove, the induction portion being inserted into the second coupling groove so that a surface of the vaporizing body and an inner surface of the induction portion are separated from each other to provide the induction passage,
the first coupling groove is formed deeper than the second coupling groove, and
the inlet passage penetrates a side surface of the heat sink and is connected to the induction passage toward the second coupling groove.
